(19) United States
(12) Patent Application Publication
(10) Pub. No.: US 2010/0130817 A1
(43) Pub. Date: May 27, 2010

(54) TISSUE MANIPULATION DEVICES

(75) Inventor: Sean P. Conlon, Loveland, OH (US)

Correspondence Address: K&L Gates LLP, 210 SIXTH AVENUE, PITTSBURGH, PA, 15222-2613, US

(73) Assignee: Ethicon Endo-Surgery, Inc., Cincinnati, OK (US)

(21) Appl. No.: 12/277,975

(22) Filed: Nov. 25, 2008

(51) Int. Cl. A61B 1/018; B65B 55/12

(52) U.S. Cl. 600/106; 53/428

(57) ABSTRACT

Devices are provided for manipulating tissue during a surgical procedure. In certain embodiments, an end effector is operably coupled to the end of an elongate shaft. The end effector has at least one tissue support linkage movably coupled thereto such that upon application of a first actuation force thereto, the tissue support linkage moves laterally outward from within the end effector to enable the surgeon to manipulate/support adjacent tissue therewith. Upon application of another actuation force to the tissue support linkage, the tissue support linkage is caused to move substantially completely within the outer perimeter of the end effector to enable the end effector to be inserted through a lumen/opening or passageway. In various embodiments, the end effector may be selectively articulateable relative to the elongate shaft.

### FIELD OF THE INVENTION

The present invention relates to methods and devices for manipulating tissue during a laparoscopic surgical procedure.

### BACKGROUND OF THE INVENTION

In laparoscopic surgical procedures, a small incision is made in the body and an elongate shaft of a surgical device is inserted through the incision to position a distal end of the shaft at a surgical site. In endoscopic procedures, the elongate shaft of a surgical device is inserted through a natural orifice, such as the mouth or anus, and is advanced along a pathway to position a distal end of the device at a surgical site. Endoscopic procedures typically require the use of a flexible shaft to accommodate the tortuous pathway of the body lumen, whereas rigid shafts can be used in laparoscopic procedures. These tools can be used to engage and/or treat tissue in a number of ways to achieve a diagnostic or therapeutic effect.

During many current laparoscopic procedures it often becomes necessary to move adjacent “non-target” tissue away from the target tissue to facilitate manipulation and actuation of the surgical instruments on the target tissue without being hampered by non-target tissue and without injuring the non-target tissue. Such challenges may be more pronounced, for example, when performing procedures within a body lumen wherein portions of the walls of the lumen may tend to collapse and hamper manipulation of the surgical instruments.

Accordingly, there remains a need for improved methods and devices for manipulating tissue during laparoscopic and other surgical procedures.

### SUMMARY OF THE INVENTION

Devices are provided for manipulating tissue during a surgical procedure. In one embodiment, a surgical device is provided that has an elongate shaft that has proximal and distal ends. Anan elongate end effector is operably coupled to the distal end of the elongate shaft. The elongate end effector has an outer perimeter that may be sized to extend through a lumen. A distal slider member is movably supported within the elongate end effector and is selectively axially movable therein in a proximal direction and a distal direction. At least one tissue support linkage is movably coupled to a portion of the end effector and the distal slider member. The tissue support linkage is selectively movable between a first position wherein the at least one tissue support linkage is substantially completely received within the outer perimeter of the elongate end effector and at least one other position wherein the at least one manipulator extends laterally outward beyond the perimeter of the elongate end effector.

### BRIEF DESCRIPTION OF THE DRAWINGS

The invention will be more fully understood from the following detailed description taken in conjunction with the accompanying drawings, in which:

FIG. 1 is a perspective view of an insertion portion of a surgical device having a tissue manipulation assembly of one embodiment of the present invention thereon with the tissue support linkages in expanded positions;

FIG. 2 is a top view of the surgical device depicted in FIG. 1;

FIG. 2A is another top view of the surgical device depicted in FIGS. 1 and 2 with the tissue manipulation assembly completely received within the outer perimeter of the insertion portion to enable the insertion portion to be inserted through a lumen or other opening;

FIG. 3 is a cross-sectional view of the insertion portion of the surgical device of FIGS. 1 and 2;

FIG. 4 is an enlarged cross-sectional view of a rotation joint portion of the surgical device of FIGS. 1-3;

FIG. 5 is a cross-sectional perspective view of the surgical device of FIGS. 1-4;

FIG. 6 is a cross-sectional view of the surgical device of FIG. 2 taken along line 6-6 in FIG. 2, with the second tissue support linkage omitted for clarity;

FIG. 7 is another cross-sectional view of the surgical device of FIG. 2 taken along line 7-7 in FIG. 2 with the second tissue support linkage omitted for clarity;

FIG. 8 is a perspective view of one embodiment of a handle portion of an embodiment of the present invention;

FIG. 9 is an exploded view of the handle portion shown in FIG. 8;

FIG. 10 is a cross-sectional view of articulation mechanism of the handle portion shown in FIG. 8; and

FIG. 11 is a cross-sectional view of an actuation mechanism of the handle portion shown in FIG. 8.

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The present invention generally provides methods and devices for manipulating tissue when performing various surgical procedures. The unique and novel features of the embodiments of the present invention may be employed in connection with any one of the articulatable joint arrangements disclosed in U.S. Patent Application Publication No. 2008/0147113A1, Published Jun. 19, 2008, entitled “Manually Articulating Devices” which is herein incorporated by reference in its entirety. However, as the present Detailed Description proceeds, those of ordinary skill in the art will understand that the various advantages provided by the embodiments of the subject invention and their equivalent structures may be employed in connection with a variety of different surgical devices including non-articulating surgical instruments. A person skilled in the art will further appreciate that the present invention has application in endoscopic procedures, laparoscopic procedures, and in conventional open surgical procedures, including robotic-assisted surgery.

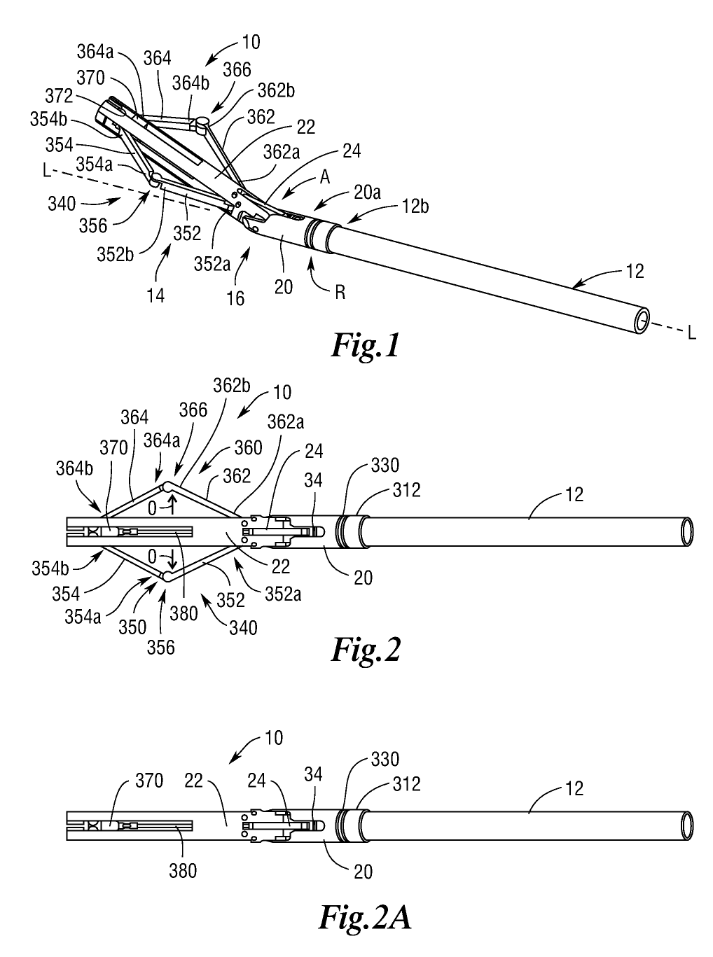

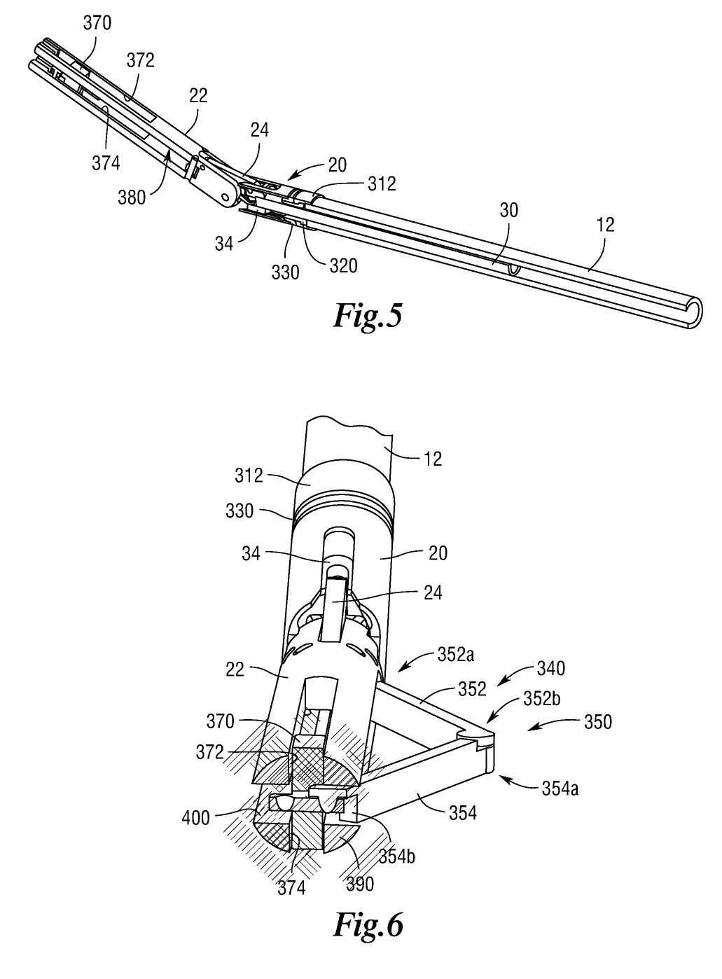

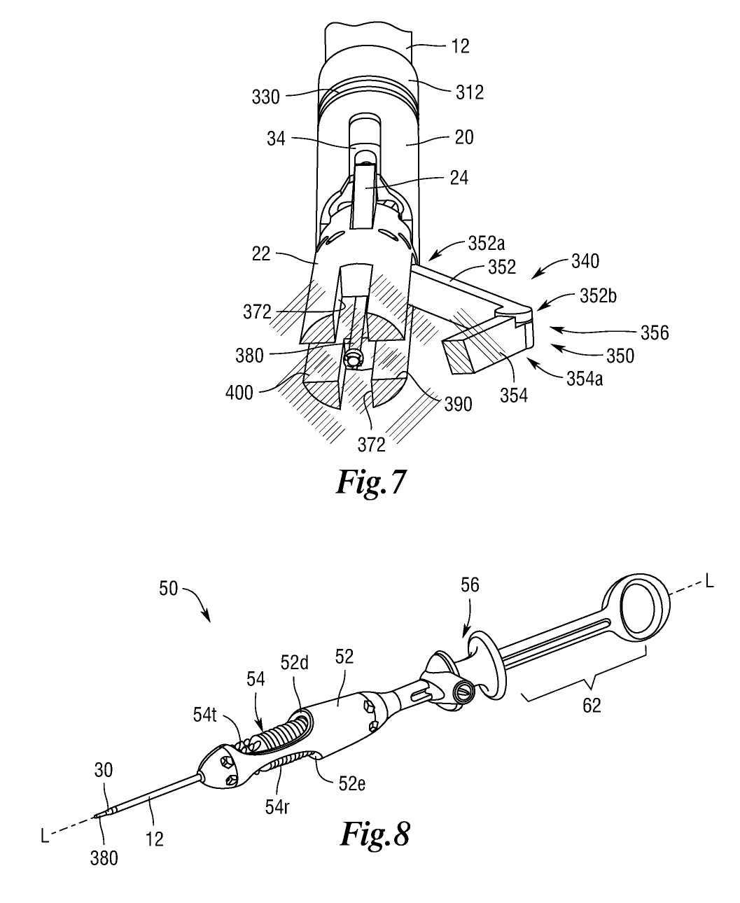

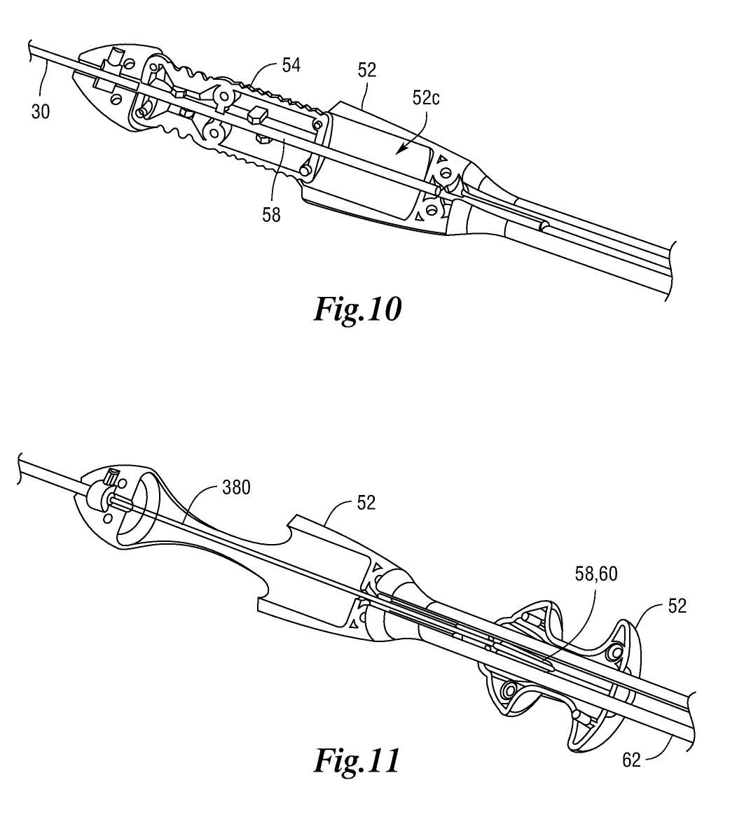

FIGS. 1-7 illustrate one exemplary embodiment of an insertion portion 10 of a manually articulating device. A handle portion of the device will be discussed in more detail below with respect to FIGS. 8-11. The insertion portion 10 is preferably configured to be inserted into a patient's body, and it can be rigid for laparoscopic applications, flexible for endoscopic applications, or it can have rigid and flexible portions as may be desired. As shown, the insertion portion 10 generally includes a hollow elongate shaft 12 having a working end or end effector 14 coupled to a distal end 12b of the elongate shaft 12 by a three-bar linkage 16. The end effector 14 may be coupled to the distal end 12b of the elongate shaft 12 by a three bar linkage arrangement of the type and construction described in detail in U.S. Patent Application Publication No. 2008/0147113A1, which has been herein incorporated by reference. The three-bar linkage 16 allows the end effector 14 to be oriented at an angle relative to a longitudinal axis L-L of the elongate shaft 12. The device can also optionally be configured to allow the end effector 14 to rotate relative to and about the longitudinal axis L-L of the elongate shaft 12. The three-bar linkage 16 is rotatably coupled to the distal end 12b of the elongate shaft 12, and thus the three-bar linkage 16, as well as the end effector 14 coupled thereto, can be positioned in various axial orientations. The location of the rotation joint R proximal of the articulation joint A is particularly advantageous in that rotation of the end effector 14 can change the location of the plane within which the end effector 14 articulates.

The three-bar linkage 16 can have a variety of configurations, but in an exemplary embodiment, it includes three links 20, 22, 24 that are pivotally coupled to one another. Each link can have a variety of configurations, but in an exemplary embodiment, the first and second links 20, 22 each comprise a clevis arrangement and the third link 24 is in the form of an elongate rod or bar. The first link 20 can have a proximal end 20a that is coupled to a distal end 12b of the elongate shaft 12 by a rotatable coupling arrangement, which will be discussed in more detail below. The distal end 20b of the first link 20 can be pivotally coupled to a proximal end 22a of the second link 22, e.g., by a pivot joint. The second link 22 comprises a portion of and provides a means for pivoting the end effector 14 relative to axis L-L. The third link 24 can extend at least partially through the first and second links 20, 22, and it can have a distal end 24b that is pivotally coupled to the second link 22, e.g., by a pivot pin, to form a three-bar linkage mechanism. The particular location at which the third link 24 mates to the second link 22 can vary, but it is preferably pivotally mated at a location that will allow the third link 24 to apply a force to the second link 22 to cause the second link 22 to articulate relative to the first link 20. The proximal end 24a of the third link 24 can be coupled to an articulation actuator 30 extending through the elongate shaft 12 and at least partially through the first link 20. The articulation actuator 30 can have a variety of configurations, but in an exemplary embodiment the articulation actuator 30 is in the form of a hollow elongate shaft or tube. Such a configuration allows an actuation wire 32 to extend therethrough for actuating the tissue manipulator assembly 300, as will be discussed below. FIG. 4 also illustrates an articulation coupling 34 for connecting the articulation actuator 30 to the third link 24. The coupling 34 may comprise a tubular member that fixedly mates to the articulation actuator 30 and pivotally mates to the third link 34. A person skilled in the art will appreciate that the articulation actuator 30 can be otherwise directly mated to the third link 24.

In use, proximal movement of the articulation actuator 30 relative to and along the longitudinal axis L-L of the elongate shaft 12 will apply a proximally-directed force to the third link 24. The third link 24 will thus apply a proximally-directed force to the second link 22, causing the second link 22 to pivot laterally relative to the longitudinal axis L-L of the elongate shaft 12. As a result, the second link 22, which comprises a portion of eth end effector 14, will move laterally in a single plane to allow the end effector 14 to extend at an angle relative the longitudinal axis L-L of the elongate shaft 12, as shown in FIG. 1. The end effector 14 can be returned to the original, longitudinally-aligned position, by moving the articulation actuator 30 distally relative to the elongate shaft 12.

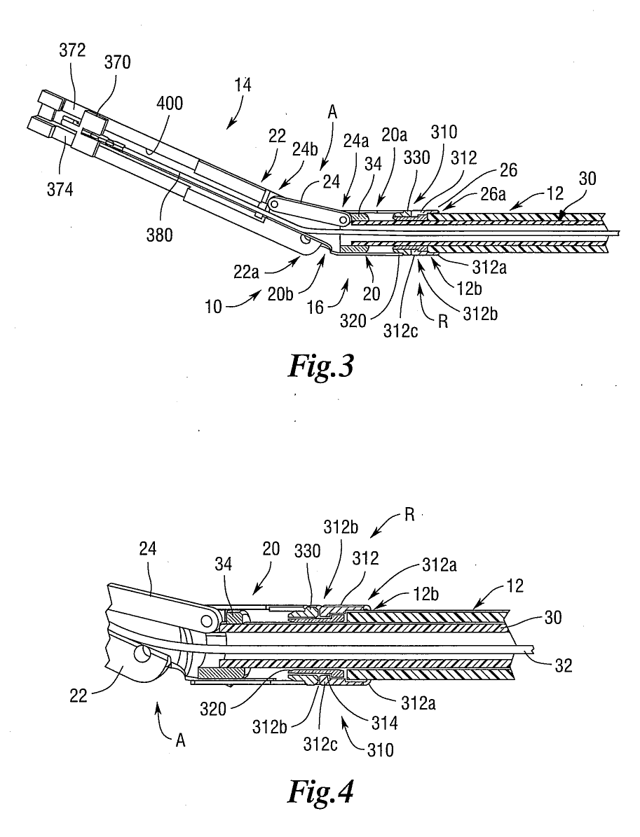

As previously indicated, in addition to articulating movement, the end effector 14 can also be configured to rotate relative to the elongate shaft 12, thus allowing the end effector 14 to be positioned in multiple angular orientations. The particular location of the rotation joint R can vary, and it can be located proximal to the three-bar linkage 16, at a mid-portion of the three-bar linkage 16, or distal to the three-bar linkage 16. In an exemplary embodiment, the rotation joint R is located proximal to the three-bar linkage 16, and more preferably proximal to the articulation joint A formed between the first and second links 20, 22. As shown in FIGS. 3 and 4, the first link 20 can be rotatably coupled to the distal end 12b of the elongate shaft 12 by a rotation coupling assembly, generally designated as 310. In various embodiments, the rotation coupling assembly 310 includes a capture ring 312 that has a proximal end 312a that is fixedly mated to the distal end of the elongate shaft 12. As can be seen in FIGS. 3 and 4, the capture ring 312 may further have a distal end 312b that has deflectable tabs 312c formed therearound. The tabs 3126c can be formed by longitudinally-extending cut-outs formed in and spaced radially around the distal end 312b of the capture ring 312. Each tab 312c can include an annular flange or lip 314 formed on an inner surface thereof.

The rotation coupling assembly 310 may further include an inner housing coupling ring 320 that is supported on the articulation actuator 30. More specifically, the inner housing coupling ring 320 has a passage 322 therethrough that is sized to enable the articulation actuator to freely move therethrough and also facilitate the free rotation of the inner housing coupling 320 about the articulation actuator 30 and axis L-L. The rotation coupling assembly 310 may further include a coupling bushing 330 that is attached to the inner housing coupling ring 320 as well as the first link 20. Those of ordinary skill in the art will appreciate that such arrangement permits the elongate shaft 12 and the capture ring 312 to rotate relative to the first link 20 and coupling bushing 330 about longitudinal axis L-L.

Rotation of the articulation actuator 30 relative to and about the longitudinal axis L-L of the elongate shaft 12 will rotate the third link 24, which is coupled to the second link 22, which in turn is coupled to the end effector 14 and the first link 20. As a result, the entire three-bar linkage 16 will rotate with the end effector 14 relative to and about the longitudinal axis L-L of the elongate shaft 12. Rotation can also be done while the end effector 14 is articulated, thereby changing the plane within which the end effector 12 articulates.

Various embodiments of the subject invention are equipped with a tissue manipulation assembly, generally designated as 340. The tissue manipulation assembly 340 may include at least one tissue support linkage that may be selectively moved relative to the second link 22 to position/manipulate adjacent tissue. In the illustrated embodiment, the tissue manipulation assembly 340 includes a first tissue support linkage 350 and a second tissue support linkage 360. As will be discussed in further detail below, the first and second tissue support linkages 350, 360 may be selectively moved from a first position wherein the support linkages 350, 360 are completely received within the outer perimeter of the second link 22 (FIG. 2A) to enable the end effector 14 to be inserted through a lumen such as, for example, a working channel of an endoscope or other opening/passage to other expanded positions wherein the tissue support linkages 350, 360 protrude laterally out of the second link 22 (FIG. 2).

The tissue support linkages 350, 360 may be actuated by axially moving a distal slider member 370 as will be discussed in further detail below. The first tissue support linkage 350 may comprise a first two bar linkage assembly that comprises a first proximal link 352 that has a proximal end 352a that pivotally coupled to the second link 22. In the illustrated embodiment, the proximal end 352a of the first proximal link 352 is pinned to the second link 22. The distal end 352b of the first proximal link 352 is pivotally coupled to a proximal end 354a of a first distal link 354 forming a first pivot joint 356. The distal end 354b of the first distal link 354 is pivotally coupled to distal slider member 370. Similarly, the second tissue manipulator 360 may comprise a second two bar linkage assembly that includes a second proximal link 362 that has a proximal end 362a that pivotally coupled to the second link 22. In the illustrated embodiment, the proximal end 362a of the second proximal link 362 is pinned to the second link 22. The distal end 362b of the second proximal link is pivotally coupled to a proximal end 364a of a second distal link 364 to form a second joint 366. The distal end 364b of the second distal link 364 is also pivotally coupled to distal slider member 370.

The particular configuration of the distal slider member 370 can vary, but in an exemplary embodiment, the distal slider member 370 has a generally rectangular configuration and is slidably disposed within and between opposed slots 372, 374 formed in a distal portion of the second link 22. Such a configuration will prevent independent rotation of the distal slider member 370 relative to the second link 22. The axial actuation of the distal slider member 370 is controlled by an actuation wire 380. Actuation wire 380 can have a variety of configurations, but in an exemplary embodiment, it is an elongate flexible cable or wire that extends through second link 22, the articulating coupling 34 which is disposed within the second link 22, and the articulation actuator 30. Actuation wire 380 is sufficiently stiff such that, upon application of an actuation force in the distal direction thereto, the actuation wire 380 causes the distal slider member 370 to move in the distal direction, yet the actuation wire 380 is sufficiently flexible to enable the actuation wire 380 to flex with the elongate shaft 12.

In use, proximal movement of the actuation wire 380 relative to the elongate shaft 12 will pull the distal slider member 370 proximally within the slots 372, 374 formed in the second link 22. The distal links 354, 364 will thus be pulled proximally and cause the first joint 356 and the second joint 366 to simultaneously move laterally outward from the second link 22 as shown in FIGS. 1 and 2 (represented by arrow “O” in FIG. 2). Conversely, distal movement of the actuation wire 380 will move the distal slider member 370 distally, which will cause the joints 356, 366, as well as the links 352, 354, 362, 364 to move inwardly into corresponding slots 390, 400, respectively in the second link 22 to the first position to enable the end effector 14 to be deployed through a lumen or other opening/passage. See FIG. 2A.

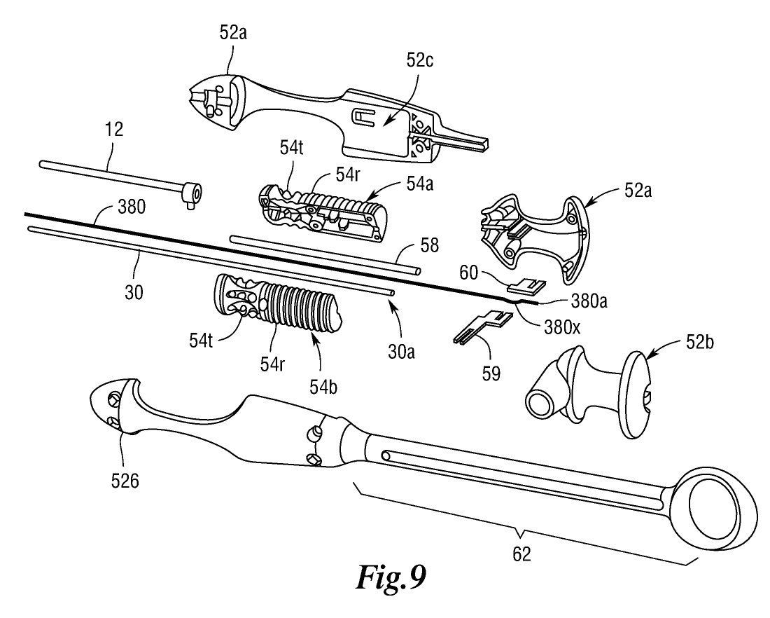

As previously indicated, the device 10 can also include a handle coupled to the proximal end of the elongate shaft and having various controls formed thereon for controlling and manipulating the device. A person skilled in the art will appreciate that the particular configuration of the handle can vary, and that various techniques known in the art can be used for effecting movement of various portions on the device. FIGS. 8-11 illustrate one exemplary embodiment of a handle 50 for use with the insertion portion 10 of the device shown in FIGS. 1-7. As shown, the handle 50 has a generally elongate cylindrical configuration to facilitate grasping thereof. The handle housing 52 can have an integral or unitary configuration, or it can be formed from two housing halves 52a, 52b that mate to enclose various components therein. The housing halves 52a, 52b are shown in FIG. 9. The various component disposed within the handle housing 52 can also vary, but in an exemplary embodiment the handle includes an articulation knob 54 for articulating and rotating the end effector 14, and an actuation knob 56 for actuating the manipulation end effector 14.

The articulation knob 54 is shown in more detail in FIGS. 3B and 3C, and as shown the knob 54 has a generally cylindrical configuration. The knob 54 can have an integral or unitary configuration, or it can be formed from two halves 54a, 54b that mate together, as shown. The proximal end 30a of the articulation actuator 30 can mate to the knob 54 such that rotation and translation of the knob 54 will cause corresponding rotation and translation of the articulation actuator 30, thereby rotating and articulating the end effector 14, as previously described. While various techniques can be used to mate the articulation actuator 30 to the articulation knob 54, in an exemplary embodiment the articulation knob 54 includes an axle 58 fixedly disposed therein and engaged between the knob halves 54a, 54b. The articulation actuator 30 extends through an inner lumen of the axel 58 and is fixedly mated thereto. Various mating techniques can be used to mate the articulation actuator 30 to the axel 58 including, for example, an interference, or compression fit, an adhesive, or other mechanical or chemical mating techniques known in the art.

In order to translate and rotate the articulation knob 54, the handle housing 52 can include an elongate cavity 52c (FIG. 3B) formed therein that slidably and rotatably receives the knob 54. The handle housing 52 can also include one or more cut-outs formed therein for allowing a user to access the knob. FIG. 3A illustrates opposed cut-outs 52d, 52e formed in the handle housing 52. The articulation knob 54 can also include features to facilitate movement thereof. For example, the articulation knob 54 can include one or more surface features formed on an external surface thereof for allowing the user to more easily grasp the knob. In the illustrated embodiment, the knob 54 includes a series of ridges 54r formed therein, as well as a series of longitudinally-oriented teeth 54b formed on a portion thereof. The 54r ridges are for a detent feature to maintain the position of the articulation. The detent snap is located in the 52c cavity.

In use, the knob 54 can be grasped by a user and rotated about its longitudinal axis (i.e., about the longitudinal axis L of the shaft 12 and handle 50). Rotation of the knob will cause corresponding rotation of the axel 58 and the articulation actuator 30. The actuation wire 32, which extends through the articulation actuator 30, will not rotate with the articulation actuator 30 since it is not coupled thereto. As previously explained, rotation of the articulation actuator 30 will cause corresponding rotation of the three-bar linkage 16 and the end effector 14 coupled thereto. The articulation knob 54 can also be slid or translated longitudinally along its axis L, and within the elongate cavity 52c formed in the handle housing 52. Proximal movement of the articulation knob 54 within the handle housing 52 will pull the articulation actuator 30 proximally, thereby articulating the end effector 14, as previously explained. Distal movement of the articulation knob 54 within the handle housing 52 will in turn move the articulation actuator 30 distally, thereby returning the end effector 14 to its original longitudinally-aligned position.

As indicated above, the device can also include an actuation knob 56 for actuating the tissue manipulation assembly 340. The actuation knob 56 can have a variety of configurations, but in the illustrated embodiment the knob 56 has a bar-bell shape. The knob 56 can have an integral or unitary configuration, or it can be formed from two halves 56a, 56b that mate together, as shown in FIG. 9. The proximal end 380a of the actuation wire 380 can mate to the actuation knob 56 such that translation of the knob 56 will cause corresponding translation of the actuation wire 380, thereby actuating the manipulation assembly 340 as previously described. While various techniques can be used to mate the actuation wire 380 to the actuation knob 56, in an exemplary embodiment the proximal end 380a of the actuation wire 380 includes a bend 380x formed therein for mating to first and second retainer members 58, 60. The retainer members 58,60, which engage the bend 380x in the actuation wire 380 therebetween, can be disposed within and mated to the actuation knob 56, as shown in FIG. 11.

In order to translate the actuation knob 56, the knob 56 can include an inner lumen extending longitudinally therethrough and it can be slidably disposed around an elongate shaft portion 62 of the handle housing 52. In use, the knob 56 can be grasped by a user and translated along the shaft portion 62 of the handle housing 52. Proximal movement of the actuation knob 56 along the shaft portion 62 will pull the actuation wire 380 proximally, thereby causing the tissue support linkages 350, 360 to laterally move out of their respective slots 390, 400 in the second link 22 to positions for supporting or moving tissue. See FIGS. 1 and 2. Distal movement of the actuation knob 56 along the shaft portion 62 will in turn move the actuation wire 380 distally to apply a distal pushing motion to the distal slider 370 to thereby move the tissue support linkages 350, 360 to their closed (first) positions (FIG. 2A) wherein the tissue support linkages 350, 360 are substantially completely received within the outer perimeter of the end effector 14. As used herein, the term “substantially completely received within the outer perimeter of the end effector” means that the tissue support linkages are received within their corresponding slots sufficiently to enable the end effector to be inserted through the lumen, opening, passageway through which it is to be used.

As indicated above, the various devices disclosed herein for manipulating tissue can be used in a variety of surgical procedures, including endoscopic procedures, laparoscopic procedures, and in conventional open surgical procedures, including robotic-assisted surgery. In one exemplary endoscopic procedure, an elongate shaft of a surgical device, such as one previously disclosed herein, can be inserted through a natural orifice and a body lumen to position an end effector located at a distal end of the elongate shaft adjacent to tissue to be treated. An articulation actuator can be translated along a longitudinal axis of the elongate shaft to cause a three-bar linkage to laterally articulate the end effector in a direction substantially perpendicular to a longitudinal axis of the elongate shaft to allow the end effector to be angularly oriented relative to the elongate shaft. This can be achieved by actuating one or more actuation mechanisms formed on a handle of the device. The method can also include rotating the end effector relative to the elongate shaft. In one embodiment, the three-bar linkage can rotate with the end effector relative to the elongate shaft. For example, the articulation actuator can be rotated relative to the elongate shaft to rotate both the three-bar linkage and the end effector. Once the end effector is positioned as desired, the tissue support linkages 350, 360 may be extended to manipulate adjacent tissue, to support the walls of a lumen or passage, etc.

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

Preferably, the invention described herein will be processed before surgery. First, a new or used instrument is obtained and if necessary cleaned. The instrument can then be sterilized. In one sterilization technique, the instrument is placed in a closed and sealed container, such as a plastic or TYVEK bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility.

It is preferred that device is sterilized. This can be done by any number of ways known to those skilled in the art including beta or gamma radiation, ethylene oxide, steam.

One skilled in the art will appreciate further features and advantages of the invention based on the above-described embodiments. Accordingly, the invention is not to be limited by what has been particularly shown and described, except as indicated by the appended claims.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. A surgical device, comprising:
an elongate shaft having a proximal end and a distal end;
an elongate end effector operably coupled to said distal end of said elongate shaft, said elongate end effector having an outer perimeter;
a distal slider member movably supported within said elongate end effector and being selectively axially movable therein in a proximal direction and a distal direction;
at least one tissue support linkage movably coupled to a portion of said end effector and said distal slider member and being selectively movable between a first position wherein said at least one tissue support linkage is substantially completely received within said outer perimeter of said elongate end effector and at least one other position wherein said at least one tissue support linkage extends laterally outward beyond said perimeter of said elongate end effector.

2. The surgical device of claim 1 wherein said distal slider member is movable between a starting position and an ending position and wherein, when said distal slider member is in said starting position, said at least one tissue support linkage is in said first position and when said distal slider member is moved from said starting position to said ending position, said at least one tissue support linkage moves to said at least one other position.

3. The surgical device of claim 1 wherein said at least one tissue support linkage comprises:
a first two bar linkage assembly pivotally coupled to said portion of said elongate end effector and said distal slider member; and
a second two bar linkage assembly pivotally coupled to another portion of said elongate end effector and said distal slider member.

4. The surgical device of claim 3 wherein said first two bar linkage assembly comprises:
a first proximal link pivotally coupled to said portion of said elongate end effector; and
a second distal link pivotally coupled to said first proximal link and said distal slider member and wherein said second two bar linkage assembly comprises:
a second proximal link pivotally coupled to said another portion of said elongate end effector; and
a second distal link pivotally coupled to said second proximal link and said distal slider member.

5. The surgical device of claim 1 further comprising an actuator member coupled to said distal slider member and protruding from said proximal end of said elongate shaft to enable actuation motions to be applied to said distal slider member to cause said at least one tissue support linkage to move between said first position and said other positions.

6. The surgical device of claim 1 wherein said end effector is movably coupled to said distal end of said elongate shaft.

7. The surgical device of claim 6 wherein said end effector is pivotally coupled to said distal end of said elongate shaft by a three-bar linkage adapted to laterally articulate relative to a longitudinal axis of the elongate shaft to allow the end effector to be angularly oriented relative to the elongate shaft.

8. The surgical device of claim 6, wherein the three-bar linkage is rotatably coupled to the elongate shaft such that the three-bar linkage and the end effector coupled thereto are adapted to rotate about a longitudinal axis of the elongate shaft.

9. The surgical device of claim 7, wherein the three-bar linkage comprises:
a first articulating link having a proximal end coupled to the distal end of the elongate shaft;
a second articulating link having a proximal end pivotally coupled to a distal end of the first articulating link, and a distal end coupled to the end effector; and
a third articulating link having a proximal end pivotally coupled to an articulation actuator extending through the elongate shaft, and a distal end pivotally coupled to the second articulating link.

10. The surgical device of claim 9, wherein the articulation actuator is adapted to translate along a longitudinal axis of the elongate shaft to laterally articulate the second link and the end effector relative to the first link.

11. The surgical device of claim 9, wherein the articulation actuator comprises a hollow elongate tube.

12. The surgical device of claim 9, wherein the articulation actuator is rotatable relative to the elongate shaft such that rotation of the articulation actuator rotates the three-bar linkage and the end effector relative to the elongate shaft.

13. The surgical device of claim 1, wherein the elongate shaft is flexible.

14. A method for processing the surgical device of claim 1 for surgery, comprising:
obtaining the surgical device of claim 1;
sterilizing the surgical device; and
storing the surgical device in a sterile container.

15. A surgical method, comprising:
obtaining the surgical device of claim 1;
ensuring that the at least one tissue support linkage is in the first position;
inserting the end effector and elongate shaft through a body lumen to position the end effector adjacent to non-target tissue;
moving the at least one tissue support linkage to one of the at least one other positions such that the at least one tissue support linkage extends laterally outward beyond the perimeter of said elongate end effector; and
manipulating the end effector to cause the at least one tissue support linkage to support at least some of the non-target tissue.

16. The surgical method of claim 15 wherein said moving the least one tissue support linkage to one of the at least one other positions comprises moving the distal slider member in a proximal direction.

17. The surgical method of claim 16 wherein said moving the distal slider member in a proximal direction comprises applying a pulling motion to the distal slider member with an actuation member attached to the distal slider member and extending through the elongate shaft.

18. The surgical method of claim 15 wherein said ensuring comprises applying a pushing motion to the distal slider member with an actuation member attached to the distal slider member and extending through the elongate shaft.

19. The surgical method of claim 15 wherein said manipulating comprises articulating the end effector relative to the elongate shaft.

20. The surgical method of claim 15 wherein said manipulating comprise rotating the end effector relative to the elongate shaft about a longitudinal axis defined by the elongate shaft.
applying an actuation motion to the at least one tissue support linkage
